(19) United States
(12) Patent Application Publication
(10) Pub. No.: US 2020/0222940 A1
(43) Pub. Date: Jul. 16, 2020

(54) Ultrasonic Transducer, Manufacturing Method Thereof, and Ultrasonic Imaging Apparatus

(71) Applicant: Hitachi, Ltd., Tokyo (JP)

(72) Inventors: Masakazu KAWANO, Tokyo (JP); Taiichi TAKEZAKI, Tokyo (JP); Shuntaro MACHIDA, Tokyo (JP); Daisuke RYUZAKI, Tokyo (JP)

(21) Appl. No.: 16/741,145

(22) Filed: Jan. 13, 2020

(30) Foreign Application Priority Data
Jan. 15, 2019 (JP) 2019-004613

(51) Int. Cl. B06B 1/02; A61B 8/00

(52) U.S. Cl. CPC B06B 1/0292; B06B 2201/76; A61B 8/4483

(57) ABSTRACT

An object of the present invention is to provide an ultrasonic transducer having a high sensitivity and a high durability. An ultrasonic transducer includes: a pair of upper and lower electrodes; a cavity layer having a vibration space directly sandwiched between the pair of electrodes; and an insulating layer sandwiched between the pair of electrodes and disposed around the vibration space. A vertical thickness of the insulating layer is greater than that of the cavity layer.

### INCORPORATION BY REFERENCE

The present application claims priority from Japanese patent application JP-2019-004613 filed on Jan. 15, 2019, the content of which is hereby incorporated by reference into this application.

### BACKGROUND OF THE INVENTION

### Field of the Invention

The present invention relates to a technique for manufacturing an ultrasonic transducer, and particularly to a technique effectively applied to a structure of the ultrasonic transducer manufactured by MEMS (Micro Electro Mechanical System) technology.

### Background Art

The ultrasonic transducer is used in various applications such as an ultrasonic imaging apparatus that inspects a human body or an internal structure of an object by transmitting and receiving an ultrasonic wave.

Conventionally, the ultrasonic transducer using vibration of a piezoelectric body has been used. However, due to recent advances in MEMS technology, a capacitive detection type ultrasonic transducer (CMUT: Capacitive Micromachined Ultrasonic Transducer) in which a vibration unit is provided on a silicon substrate has been developed.

The CMUT has a structure in which upper and lower electrodes are arranged with a cavity layer therebetween, and is a vibration element using the fact that an electrostatic force is generated in a membrane over a cavity by applying a voltage between the upper electrode and the lower electrode to generate a potential difference. The ultrasonic wave is transmitted by vibrating the membrane by temporally changing the voltage applied to the upper and lower electrodes, and is received by detecting displacement of the membrane as a voltage change or a current change with a constant voltage applied between the upper and lower electrodes.

For example, in the CMUT disclosed in Japanese Patent No. 4,869,593, JP-A-2016-537083, and Japanese Patent No. 5,859,056, the lower electrode, the cavity layer, the upper electrode, and an insulating film are laminated in this order on the substrate. In the CMUTs, the cavity layer is directly sandwiched between the upper and lower electrodes, and no insulating layer is disposed between the upper electrode and the cavity layer or between the lower electrode and the cavity layer. However, the insulating layer for supporting the insulating film over the upper electrode from its lower surface is disposed to surround a periphery of the cavity layer except for above and below the cavity layer. The insulating layer for supporting the insulating film is disposed at a position where a plurality of CMUT elements on the substrate are connected, and is shared by the plurality of CMUT elements.

### SUMMARY OF THE INVENTION

It is known that sensitivity of the ultrasonic transducer is increased as a distance between the upper and lower electrodes is reduced. In the ultrasonic transducer in which the cavity layer is directly sandwiched between the upper and lower electrodes, such as the CMUT of Japanese Patent No. 4,869,593, JP-T-2016-537083, and Japanese Patent No. 5,859,056, since the insulating layer is not disposed between the upper electrode and the cavity layer or between the lower electrode and the cavity layer, the distance between the upper and lower electrodes can be reduced. However, in such a CMUT, when the distance between the upper and lower electrodes is reduced, an electric field strength between the upper and lower electrodes of the insulating layer disposed to surround the periphery of the cavity layer is increased, and thus there has been a possibility that the insulating layer is destroyed.

An object of the present invention is to provide an ultrasonic transducer having a high sensitivity and a high durability.

In order to achieve the above object, the present invention provides an ultrasonic transducer including: a pair of upper and lower electrodes; a cavity layer having a vibration space directly sandwiched between the pair of electrodes; and an insulating layer sandwiched between the pair of electrodes and disposed around the vibration space. A vertical thickness of the insulating layer is greater than that of the cavity layer.

According to the present invention, strength of the insulating layer can be maintained even if a thickness of the cavity layer between the upper and lower electrodes is made smaller than before. Therefore, it is possible to provide the ultrasonic transducer having a high sensitivity and a high durability.

### BRIEF DESCRIPTION OF THE DRAWINGS

FIG. 1A is a top view showing a wiring example of an ultrasonic transducer 10 of the present invention, and FIG. 1B is a partially enlarged view of a CMUT array;

FIG. 2A is a cross-sectional view of an ultrasonic transducer of Embodiment 1 taken along a line A-A in FIG. 1B, and FIG. 2B is a cross-sectional view of the ultrasonic transducer of Embodiment 1 taken along a line B-B in FIG. 1B;

FIGS. 3A to 3E are cross-sectional views showing a method for manufacturing the ultrasonic transducer of Embodiment 1;

FIGS. 4A to 4D are cross-sectional views showing the method for manufacturing the ultrasonic transducer of Embodiment 1;

FIG. 5 is a cross-sectional view of an ultrasonic transducer of Embodiment 2 taken along the line A-A in FIG. 1B;

FIGS. 6A to 6D are cross-sectional views showing the method for manufacturing the ultrasonic transducer of Embodiment 2;

FIGS. 7A to 7C are cross-sectional views showing the method for manufacturing the ultrasonic transducer of Embodiment 2;

FIG. 8 is a cross-sectional view of an ultrasonic transducer of a modification;

FIG. 9 is a cross-sectional view of an ultrasonic transducer of Embodiment 3 taken along the line A-A in FIG. 1B;

FIGS. 10A to 10F are cross-sectional views showing the method for manufacturing the ultrasonic transducer of Embodiment 3;

FIGS. 11A to 11E are cross-sectional views showing the method for manufacturing the ultrasonic transducer of Embodiment 3; and

FIG. 12 is a block diagram showing a configuration example of an ultrasonic imaging apparatus of the present invention.

### DESCRIPTION OF THE PREFERRED EMBODIMENTS

Hereinafter, an embodiment of the present invention will be described with reference to the drawings.

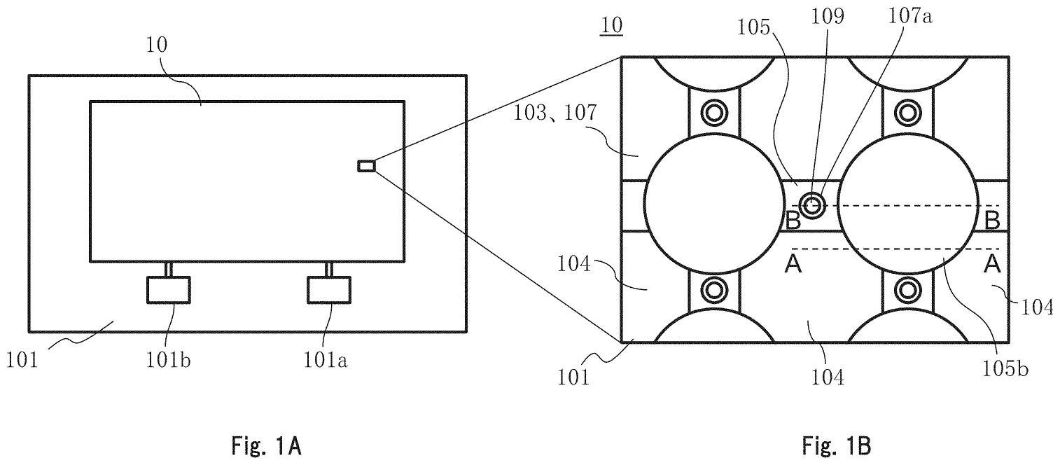

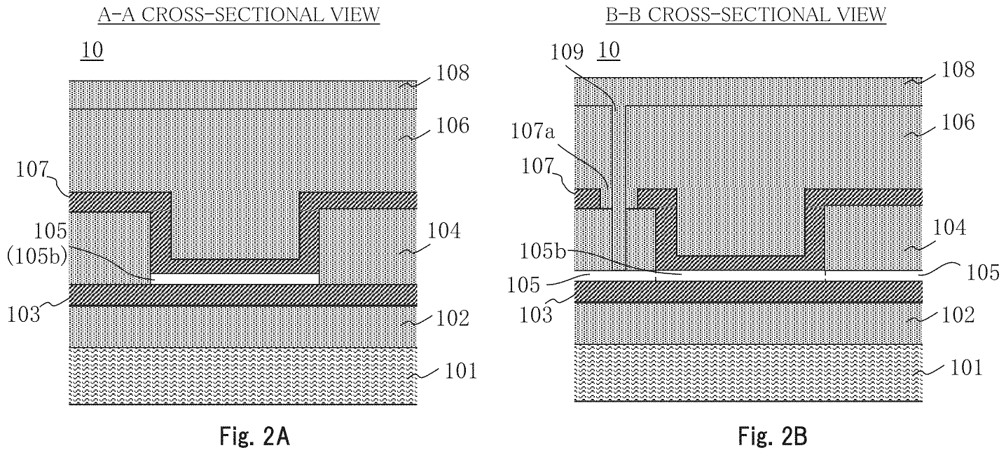

First, an outline of a configuration of an ultrasonic transducer 10 according to the present invention will be described with reference to FIGS. 1A, 1B, 2A and 2B. The ultrasonic transducer 10 is a so-called CMUT formed on a substrate 101 by MEMS technology, and may be a single element, or a CMUT array or a CMUT chip in which a large number of CMUT elements are arranged as shown in FIG. 1A.

Hereinafter, the ultrasonic transducer 10 will be described using an example in which a plurality of CMUT elements are arranged. FIG. 1B shows an example in which a part of the plurality of CMUT elements included in the ultrasonic transducer 10 is partially enlarged, FIG. 2A shows an A-A cross-sectional view of FIG. 1B, and FIG. 2B shows a B-B cross-sectional view of FIG. 1B. In FIG. 1B, the CMUT element is shown as a circle for the sake of explanation, however, this example does not limit a shape of the element.

As shown in FIG. 2A, each CMUT element of the ultrasonic transducer 10 includes: a pair of upper and lower electrodes (upper electrode 107 and lower electrode 103); a cavity layer 105 having a space (hereinafter referred to as a vibration space) 105b that is directly sandwiched between the upper and lower electrodes 107, 103 and functions as a membrane; and an insulating layer 104 that is sandwiched between the upper electrode 107 and the lower electrode 103 and disposed around the vibration space 105b of the cavity layer 105. A vertical thickness of the insulating layer 104 is greater than that of the cavity layer 105.

That is, since the upper electrode 107 and the lower electrode 103 have a region where they directly sandwich the cavity layer 105 from above and below and a region where they are arranged above and below the insulating layer 104 thicker than the cavity layer 105, a distance between the upper and lower electrodes 107 and 103 arranged above and below the insulating layer 104 is greater than the distance between the upper and lower electrodes 107 and 103 directly sandwiching the cavity layer 105.

A thickness of the cavity layer 105 is about several nm to several hundred nm, and the distance between the upper and lower electrodes 107 and 103 is also about several nm to several hundred nm in the region directly sandwiching the cavity layer 105.

On the other hand, the thickness of the insulating layer 104 is, for example, a thickness such that the electric field between the upper and lower electrodes of the insulating layer 104 is 200 V/μm, and when a driving voltage of the ultrasonic transducer 10 is 100 V, a film thickness of an insulating film is 500 nm. The thickness of the insulating layer 104 is preferably 5 times or more the thickness of the cavity layer 105.

As described above, since the ultrasonic transducer 10 has the vibration space 105b directly sandwiched between the upper and lower electrodes 107 and 103, the distance between the upper and lower electrodes 107 and 103 can be made smaller than before, and sensitivity of the ultrasonic transducer 10 is high. Further, even if the distance between the upper and lower electrodes 107 and 103 arranged above and below the cavity layer 105 is made smaller than before, the thickness of the insulating layer 104 can be maintained. Therefore, even if the voltage between the electrodes 107 and 103 arranged above and below the insulating layer 104 is increased, an electric field strength of the insulating layer 104 can be kept low. Thus, it is possible to provide the ultrasonic transducer having a high sensitivity and a high durability.

In the ultrasonic transducer 10, the CMUT elements having the above-described configuration are arranged on the substrate 101 made of a semiconductor substrate such as single crystal silicon. As shown in FIG. 1A, an upper electrode pad 101a and a lower electrode pad 101b are arranged on the substrate 101. The upper electrode 107 is connected to the upper electrode pad 101a, and the lower electrode 103 is connected to the lower electrode pad 101b.

Further, as shown in FIG. 1B, the cavity layer 105 for connecting the plurality of CMUT elements extends around each CMUT element other than above and below the vibration space 105b. As shown in FIG. 2B which is a B-B cross-sectional view of FIG. 1B, at a position where the plurality of CMUT elements are connected by the cavity layer 105, the cavity layer 105 is sandwiched between a lower surface of the insulating layer 104 and an upper surface of the lower electrode 103. Since this portion has a long inter-electrode distance and a small electrostatic force in addition to high rigidity, it does not contribute to vibration as the membrane during driving.

In a portion of the cavity layer 105 extending from the vibration space 105b, there is an embedded portion in which a vertical through-hole 109 formed when the cavity layer 105 is formed in a process for manufacturing the CMUT element is embedded with the insulating material. A portion of the upper electrode 107 corresponding to the through-hole 109 (embedded portion) is a non-electrode region 107a where no electrode layer is formed.

Inside of the cavity layer 105 and the vibration space 105b is a vacuum, and a region such as the upper electrode 107 disposed above the vibration space 105b is the membrane that vibrates during driving. A resonance frequency of the membrane is determined by an area of the vibration space 105b.

Here, operation of the ultrasonic transducer 10 will be briefly described. First, when a drive signal is applied between the upper and lower electrodes 107 and 103, a region disposed above the vibration space 105b vibrates as the membrane. Thus, an ultrasonic wave is transmitted toward an inspection object. When an acoustic signal transmitted from the membrane and reflected inside the inspection object reaches the membrane, the membrane vibrates and the acoustic signal is converted into an electrical signal. According to information of the converted electrical signal, an ultrasonic imaging apparatus described below generates an image to be inspected. More detailed operation of the entire ultrasonic imaging apparatus will be described below.

### Embodiment 1

Hereinafter, the configuration of an ultrasonic transducer of Embodiment 1 will be described in detail with reference to FIGS. 2A and 2B.

An insulating layer 102 and the lower electrode 103 are arranged in this order from the substrate 101 side on an upper surface of the substrate 101. The cavity layer 105 including the vibration space 105b having a flat shape and the insulating layer 104 surrounding a periphery of the vibration space 105b other than above and below the vibration space 105b are arranged on the upper surface of the lower electrode 103. The upper electrode 107 is disposed to cover the insulating layer 104 and the vibration space 105b. A height of the insulating layer 104 is higher than that of the vibration space 105b, and the upper electrode 107 has a stepped shape in which a portion disposed on an upper surface of the vibration space 105b is closer to the lower electrode 103 than a portion disposed on an upper surface of the insulating layer 104. On an upper surface of the upper electrode 107, insulating layers 106 and 108 are arranged in this order from the substrate 101 side.

Note that as a material of the substrate 101, glass, quartz, sapphire or the like can be used in addition to single crystal silicon.

For the upper electrode 107 and the lower electrode 103, tantalum (Ta) having a thickness of about 10 nm to 300 nm can be used. In addition to tantalum, molybdenum (Mo), tungsten (W), gold (Au), platinum (Pt), polycrystalline silicon doped with impurities at a high concentration, amorphous silicon, indium tin oxide (ITO), or the like can be used. The upper electrode 107 and the lower electrode 103 can be a single layer film or a multilayer film made of the above-mentioned materials. Note that a material which does not dissolve by an etching solution for forming the cavity layer 105 is used for the upper electrode 107 and the lower electrode 103.

The insulating layers 102, 104, 106, and 108 may be formed of the same material or different materials. Each of the insulating layers can be a single layer film or a laminated film made of one or more insulating materials selected from silicon oxide (SiO₂), silicon nitride (Si₃N₄), aluminum oxide (Al₂O₃), titanium oxide (TiO₂), zirconium oxide (ZrO₂), hafnium oxide (HfO₂), silicon-doped hafnium oxide, and the like. The insulating layers may be formed of the same material or different materials.

For the insulating layer 102, a material having good adhesion to a material of the substrate and a material of the lower electrode 103 formed thereon is selected. Since the insulating layer 104 is a portion where a high electric field is generated, a material having high voltage resistance such as SiO₂ is preferred for the insulating layer 104. The material forming the insulating layer 106 is preferably a material having high withstand voltage like the insulating layer 104. Further, since the insulating layers 106 and 108 constitute the membrane together with the upper electrode 107 and are displaced during ultrasonic transmission/reception, a shape of the membrane before displacement is flat. Therefore, it is preferred for the insulating layers 106 and 108 to stack a plurality of layers by using a material such as SiN having tensile stress and a material such as SiO₂ having compressive stress, to ensure flatness.

The thicknesses of the insulating layers can be about 10 nm to 10,000 nm for the insulating layer 102, about 10 nm to 10,000 nm for the insulating layer 106, and about 10 nm to 10,000 nm for the insulating layer 108.

### <<Method for Manufacturing Ultrasonic Transducer of Embodiment 1>>

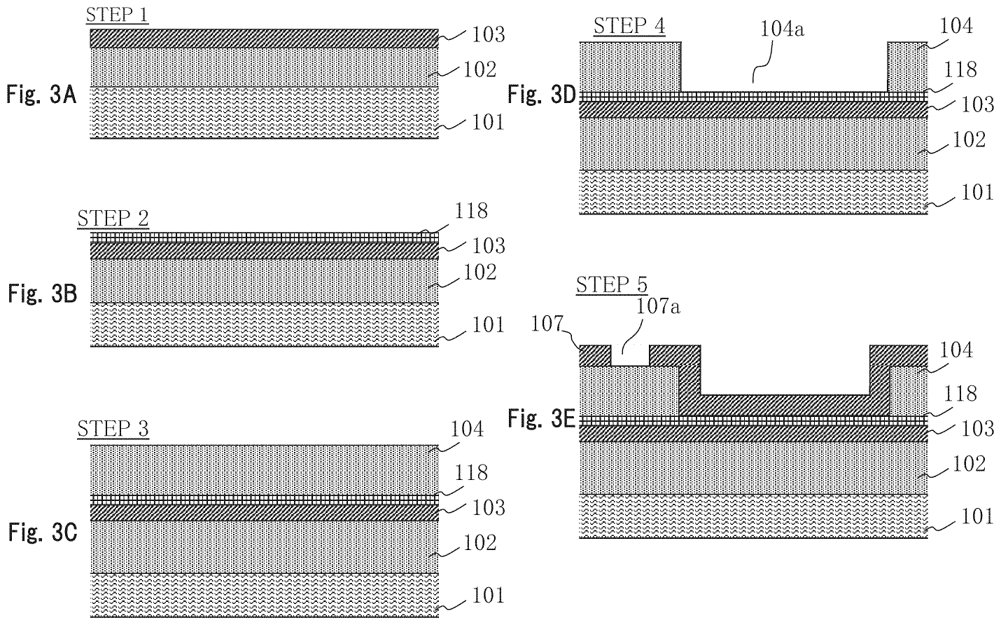

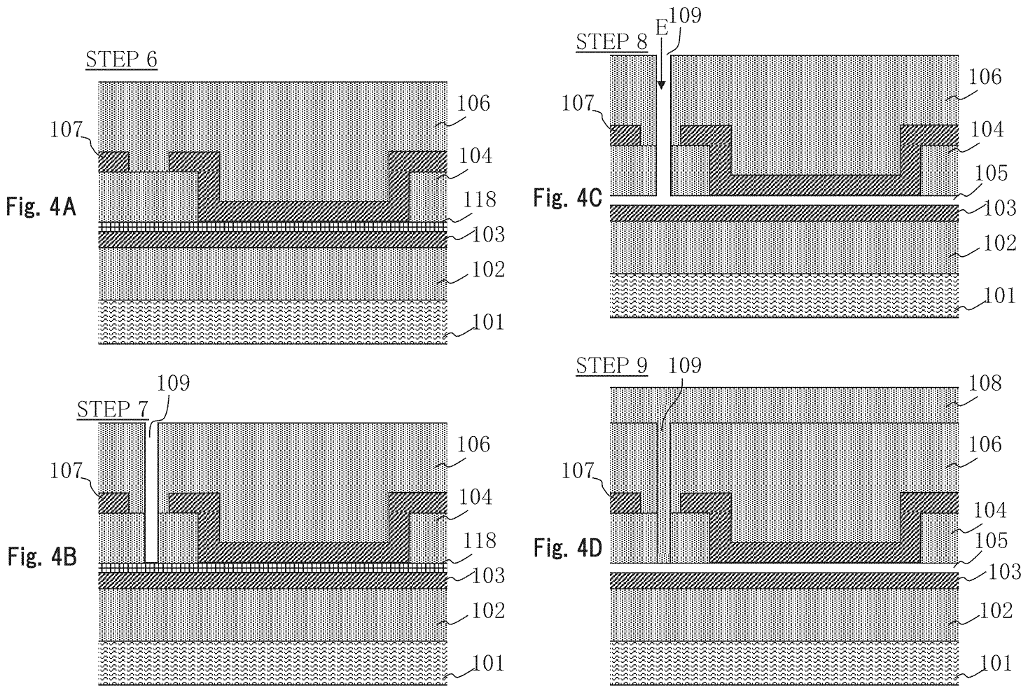

Next, a method for manufacturing the ultrasonic transducer of Embodiment 1 will be described with reference to FIGS. 3A to 3E and FIGS. 4A to 4D.

### [Step 1]

First, as shown in FIG. 3A, the insulating layer 102 and the lower electrode 103 are sequentially formed on the substrate 101. The insulating layer 102 and the lower electrode 103 can be formed by a known film forming technique such as a plasma CVD method, a vapor deposition method, or a sputtering method.

### [Step 2]

Next, as shown in FIG. 3B, a metal film (sacrificial layer) 118 is formed on the upper surface of the lower electrode 103 to have a highly uniform film thickness by the sputtering method or the like. The sacrificial layer 118 is a layer temporarily provided to form the cavity layer 105, and is later etched away by an etchant. As a material of the sacrificial layer 118, cobalt (Co) is used in consideration of easy removal by the etchant and etching selectivity. In addition, aluminum (Al), titanium (Ti), chromium (Cr) or the like can be used for the sacrificial layer 118. The thickness of the sacrificial layer 118 determines a height of the cavity layer 105.

### [Step 3]

Subsequently, as shown in FIG. 3C, the insulating layer 104 is deposited on an upper surface of the sacrificial layer 118 by the known film forming technique.

### [Step 4]

Subsequently, as shown in FIG. 3D, an opening 104a reaching the upper surface of the sacrificial layer 118 is formed in the insulating layer 104 by using a lithography method or a dry etching method. An area of the opening 104a is set according to a size of the membrane so that the membrane above the vibration space 105b has a predetermined resonance frequency.

### [Step 5]

Subsequently, as shown in FIG. 3E, after a material of the upper electrode 107 is deposited to cover the insulating layer 104 and the upper surface of the sacrificial layer 118 exposed from the opening 104a formed in Step 4, the upper electrode 107 is patterned. The upper electrode 107 is patterned to exclude a region (the non-electrode region 107a) including the vertical through-hole formed in a later step in order to form the cavity layer 105.

### [Step 6]

Subsequently, as shown in FIG. 4A, the insulating layer 106 is formed to cover the upper electrode 107 using the known film forming technique. At this time, it is preferred to embed the same insulating material as the insulating layer 106 also in the non-electrode region 107a.

### [Step 7]

Subsequently, as shown in FIG. 4B, the through-hole 109 is formed to penetrate the non-electrode region 107a from an upper surface of the insulating layer 106 and reach the sacrificial layer 118.

### [Step 8]

Subsequently, as shown in FIG. 4C, an etchant E is introduced from the through-hole 109 to dissolve the sacrificial layer 118 and form the cavity layer 105. As the etchant, one that selectively dissolves the sacrificial layer 118 without dissolving the insulating layer 104 and the electrodes around the sacrificial layer 118 can be used, and for example, hydrochloric acid can be used.

### [Step 9]

When the sacrificial layer 118 is sufficiently etched and formation of the cavity layer 105 is completed, the insulating material is deposited on the upper surface of the insulating layer 106 to form the insulating layer 108 as shown in FIG. 4D. At this time, the through-hole 109 is also preferably filled with the same insulating material as the insulating layer 108. The ultrasonic transducer of Embodiment 1 is manufactured by the manufacturing method as described above.

In such a method for manufacturing the ultrasonic transducer, since the distance between the upper and lower electrodes directly sandwiching the upper and lower sides of the cavity layer 105 can be reduced to about several tens of nanometers while the insulating layer 104 surrounding the periphery of the vibration space 105b is maintained thick, the ultrasonic transducer having a high sensitivity and a high durability can be manufactured.

### Embodiment 2

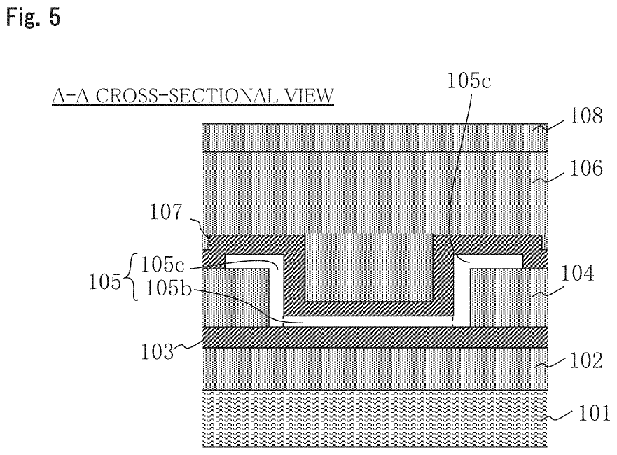

Hereinafter, regarding an ultrasonic transducer of Embodiment 2, differences from the ultrasonic transducer of Embodiment 1 will be described with reference to FIG. 5. In Embodiment 1, the ultrasonic transducer in which the cavity layer 105 has a flat shape has been described, however, in the ultrasonic transducer of Embodiment 2, the cavity layer 105 has a stepped shape.

Specifically, the cavity layer 105 of the ultrasonic transducer of Embodiment 2 has a space (hereinafter referred to as a non-vibration space) 105c along the stepped shape of the upper electrode 107 between the upper electrode 107 having a stepped shape and the insulating layer 104. The non-vibration space 105c is configured to ride on the upper surface of the insulating layer 104 on an outer peripheral side of the vibration space 105b. An outer peripheral end of the step-shaped non-vibration space 105c is covered by the upper electrode 107, and the upper electrode 107 is in contact with the upper surface of the insulating layer 104 on the outer peripheral side of the outer peripheral end of the non-vibration space 105c.

In this ultrasonic transducer, during driving, a region disposed above the vibration space 105b directly sandwiched between the upper and lower electrodes 107 and 103 out of the upper electrode 107 and the insulating layers 106 and 108 functions as the membrane. The resonance frequency of the membrane is determined by an area of the region.

### <<Method for Manufacturing Ultrasonic Transducer of Embodiment 2>>

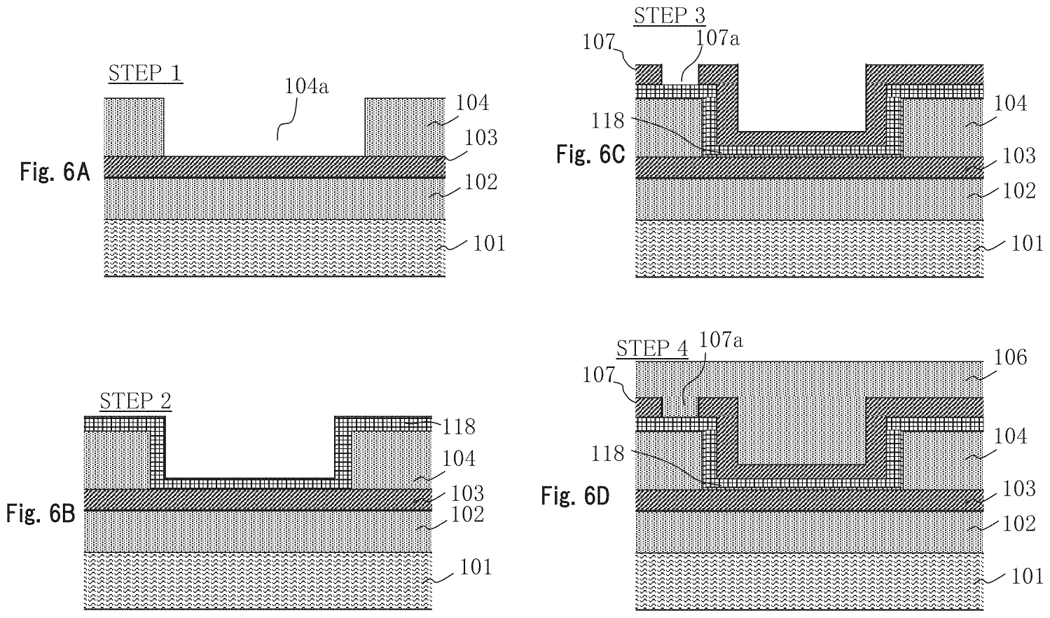

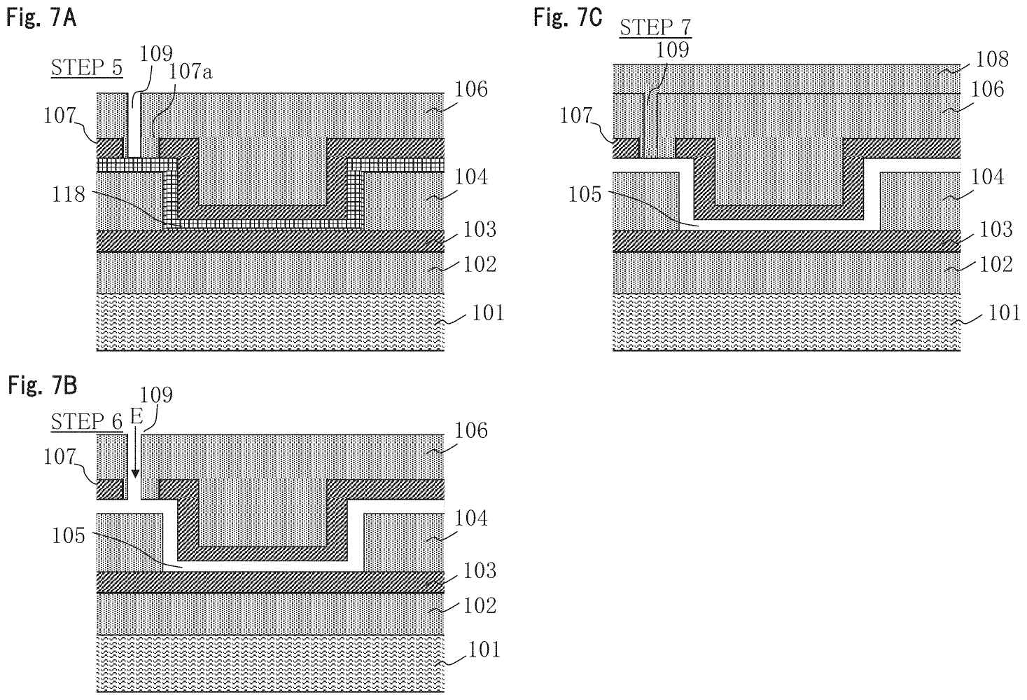

Next, the method for manufacturing the ultrasonic transducer of Embodiment 2 will be described with reference to FIGS. 6A to 6D and FIGS. 7A to 7C.

### [Step 1]

First, as shown in FIG. 6A, the insulating layer 102 and the lower electrode 103 are sequentially formed on the substrate 101 in the same manner as in Step 1 of Embodiment 1 described with reference to FIG. 3A. The insulating layer 104 is formed on the upper surface of the lower electrode 103 by using the same film forming technique as in Step 3 of FIG. 3C. The opening 104a reaching the upper surface of the lower electrode 103 is formed in the insulating layer 104 by using the lithography method or the dry etching method.

### [Step 2]

Next, as shown in FIG. 6B, the metal film (sacrificial layer) 118 is formed to cover the insulating layer 104 and the upper surface of the lower electrode 103 exposed from the opening 104a. The material and film thickness of the sacrificial layer 118 are the same as in Step 2 of Embodiment 1.

### [Step 3]

Subsequently, after forming the material of the upper electrode 107, as shown in FIG. 6C, the upper electrode 107 is patterned to exclude the non-electrode region 107a.

### [Step 4]

Subsequently, as shown in FIG. 6D, the insulating layer 106 is formed to cover the upper electrode 107. At this time, it is preferred to embed the same insulating material as the insulating layer 106 also in the non-electrode region 107a.

### [Step 5]

Subsequently, as shown in FIG. 7A, the through-hole 109 is formed to penetrate the non-electrode region 107a from the upper surface of the insulating layer 106 and reach the sacrificial layer 118. In Embodiment 2, since the through-hole 109 reaching the sacrificial layer 118 is formed above a region where the insulating layer 104 is formed, even if the sacrificial layer 118 is thin and the through-hole 109 penetrates the sacrificial layer 118, the insulating layer 104 acts as a stopper that prevents the embedded portion 109 from reaching the lower electrode 103.

### [Step 6]

Subsequently, as shown in FIG. 7B, the etchant E is introduced from the through-hole 109 to dissolve the sacrificial layer 118 and form the cavity layer 105 in the same manner as in Step 8 of Embodiment 1.

### [Step 7]

When the sacrificial layer 118 is sufficiently etched and the formation of the cavity layer 105 is completed, as shown in FIG. 7C, the insulating material is deposited on the upper surface of the insulating layer 106 to form the insulating layer 108. At this time, the through-hole 109 is also preferably filled with the same insulating material as the insulating layer 108. The ultrasonic transducer of Embodiment 2 is manufactured by the manufacturing method as described above.

As described above, in the method for manufacturing the ultrasonic transducer of Embodiment 2, since the through-hole 109 for etching the sacrificial layer 118 is formed above the region where the insulating layer 104 is formed, even if the sacrificial layer 118 is thin and the through-hole 109 penetrates the sacrificial layer 118, the insulating layer 104 acts as the stopper, so that the insulating layer 104 can prevent the embedded portion 109 from reaching the lower electrode 103. The insulating layer 104 also acts as the stopper that prevents the etchant introduced from the through-hole 109 from reaching the lower electrode 103 when the sacrificial layer 118 is etched to form the cavity layer 105. Therefore, in the manufacturing method, the ultrasonic transducer can be manufactured with a high yield.

In the ultrasonic transducer of Embodiment 2, since the cavity layer 105 has a stepped shape along the stepped shape of the upper electrode 107, a contact area between the insulating layer 104 and the cavity layer 105 is larger than that of the ultrasonic transducer in which the cavity layer 105 is flat and in contact with the insulating layer 104 only on an outer peripheral surface thereof. Therefore, the ultrasonic transducer of Embodiment 2 can be manufactured with a high yield by closely contacting the insulating layer 104 and the cavity layer 105.

### <Modification>

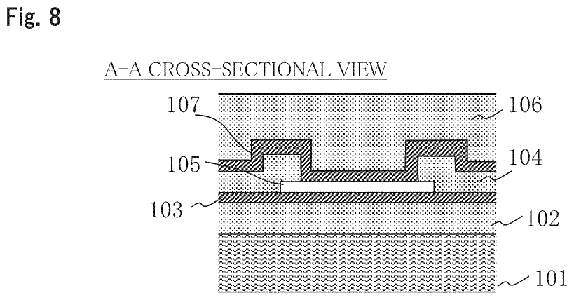

The ultrasonic transducer of Embodiment 2 has a structure in which the cavity layer 105 has a stepped shape along the stepped shape of the upper electrode 107. However, as shown in FIG. 8, the ultrasonic transducer may have a structure in which the insulating layer 104 has a stepped shape along the stepped shape of the upper electrode 107. In the ultrasonic transducer of this modification, the cavity layer 105 has a flat shape, and corners and wall surfaces of the insulating layer 104 are not located below the vibration space that vibrates during driving. Therefore, in the ultrasonic transducer, even if the upper electrode 107 vibrates during driving, it is difficult to contact the corners and side surfaces of the insulating layer 104, and driving reliability is further improved.

### Embodiment 3

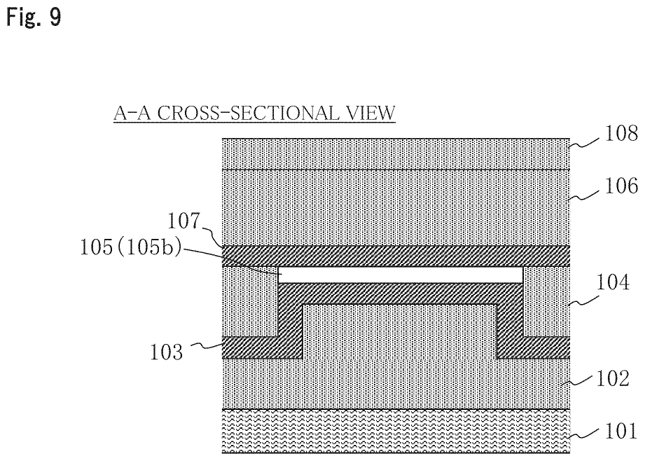

Hereinafter, an ultrasonic transducer of Embodiment 3 will be described with reference to FIG. 9. In Embodiment 1, the ultrasonic transducer in which the lower electrode 103 has a flat shape and the upper electrode 107 having a stepped shape has been described. However, in the ultrasonic transducer of Embodiment 3, the upper electrode 107 has a flat shape, and the lower electrode 103 has a stepped shape. Note that in the ultrasonic transducer of Embodiment 3, the cavity layer 105 has a flat shape like the cavity layer of the ultrasonic transducer of Embodiment 1.

Specifically, the lower electrode 103 has a region where the vibration space 105b of the cavity layer 105 is disposed on the upper surface thereof and a region where the insulating layer 104 is disposed on the upper surface thereof. Since the vibration space 105b is thinner than the insulating layer 104, the lower electrode 103 has a stepped shape in which the region where the vibration space 105b is disposed on the upper surface thereof is closer to the upper electrode 107 than the region where the insulating layer 104 is disposed on the upper surface thereof.

The membrane is usually formed in a concave shape or a convex shape due to a balance of residual stress of each layer constituting the membrane. Therefore, at the time of manufacturing the ultrasonic transducer, the residual stress of each layer is controlled to control deflection (concave or convex shape) of the membrane. Since the ultrasonic transducer of Embodiment 3 has a structure in which there is no step in the membrane and a layer above the cavity layer 105 is flat, it is easy to control the concave or convex shape of the membrane when completed by controlling the residual stress.

### <<Method for Manufacturing Ultrasonic Transducer of Embodiment 3>>

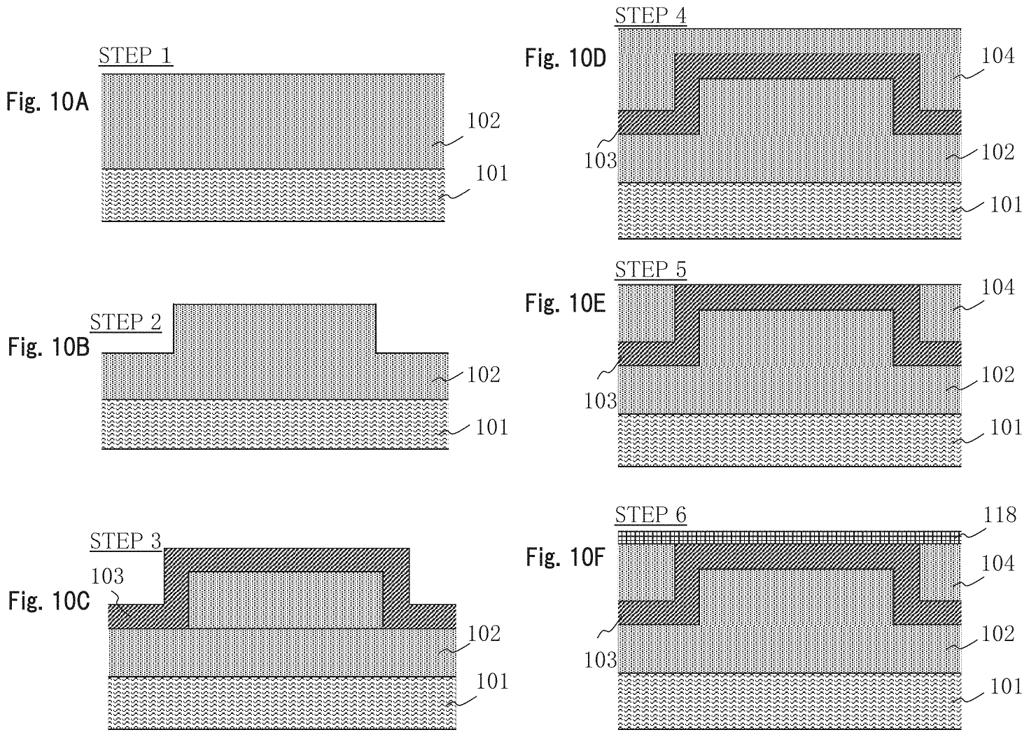

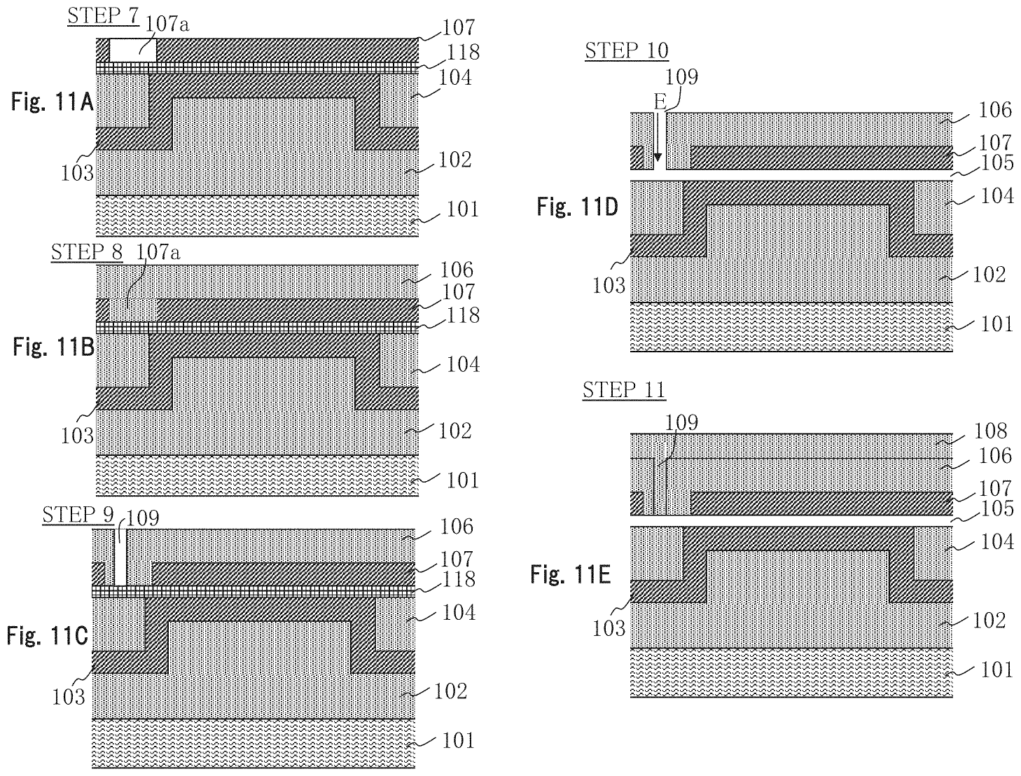

Next, the method for manufacturing the ultrasonic transducer of Embodiment 3 will be described with reference to FIGS. 10A to 10F and FIGS. 11A to 11E.

### [Step 1]

First, the insulating layer 102 is sequentially formed on the substrate 101 as shown in FIG. 10A as in Step 1 of Embodiment 1 described with reference to FIG. 3A.

### [Step 2]

Next, as shown in FIG. 10B, the insulating layer 102 is patterned into a stepped shape by using the lithography method or the dry etching method. At this time, the insulating layer 102 is patterned so that a height of a step of the insulating layer 102 is equal to the height of the insulating layer 104 formed in a later step.

### [Step 3]

Subsequently, as shown in FIG. 10C, the lower electrode 103 is deposited with a uniform film thickness so as to cover the insulating layer 102.

### [Step 4]

Subsequently, as shown in FIG. 10D, the insulating material is formed to cover an entire surface of the lower electrode 103.

### [Step 5]

Subsequently, as shown in FIG. 10E, the insulating material formed in Step 4 is etched, to form the insulating layer 104 so that an upper surface of an upper step of the stepped shape of the lower electrode 103 is exposed. Thus, the upper surface of the insulating layer 104 is flush with the upper surface of the lower electrode 103.

### [Step 6]

Subsequently, as shown in FIG. 10F, the sacrificial layer 118 is formed on the upper surfaces of the insulating layer 104 and the lower electrode 103 by sputtering or the like. The material and film thickness of the sacrificial layer 118 can be the same as in Step 2 of Embodiment 1. Since the upper surface of the insulating layer 104 formed in Step 5 is flush with the upper surface of the lower electrode 103, the sacrificial layer 118 has a flat shape.

### [Step 7]

Subsequently, after the material of the upper electrode 107 is formed on the upper surface of the sacrificial layer 118, the upper electrode 107 is patterned to exclude the non-electrode region 107a as shown in FIG. 11A.

### [Step 8]

Subsequently, as shown in FIG. 11B, the insulating layer 106 is formed to cover the upper electrode 107. At this time, it is preferred to embed the same insulating material as the insulating layer 106 also in the non-electrode region 107a.

### [Step 9]

Subsequently, as shown in FIG. 11C, the through-hole 109 is formed to penetrate the non-electrode region 107a from the upper surface of the insulating layer 106 and reach the sacrificial layer 118.

### [Step 10]

Subsequently, as shown in FIG. 11D, the etchant E is introduced from the through-hole 109 to dissolve the sacrificial layer 118 and form the cavity layer 105 in the same manner as in Step 8 of Embodiment 1.

### [Step 11]

When the sacrificial layer 118 is sufficiently etched and the formation of the cavity layer 105 is completed, as shown in FIG. 11E, the insulating material is applied to the upper surface of the insulating layer 106 to form the insulating layer 108. At this time, the through-hole 109 is also preferably filled with the same insulating material as the insulating layer 108. The ultrasonic transducer of Embodiment 3 is manufactured by the manufacturing method as described above.

Although the above has described the method for manufacturing the ultrasonic transducer by laminating components in order from the bottom on the upper surface of the substrate 101, the present invention is not limited to this. The ultrasonic transducer 10 may be formed by bonding a laminate of the components from the substrate 101 side (lower surface side) and a laminate of the components from the upper surface side.

### <Ultrasonic Imaging Apparatus>

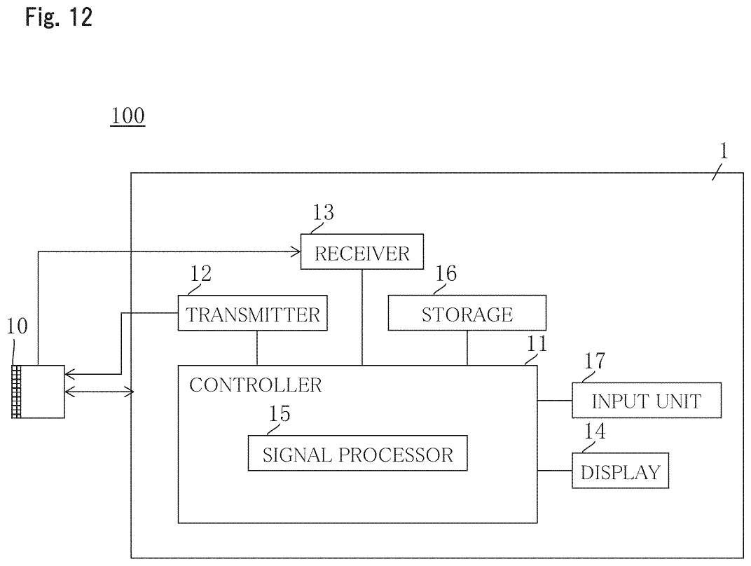

Hereinafter, an ultrasonic imaging apparatus 100 of the present invention will be described with reference to FIG. 12.

The ultrasonic imaging apparatus 100 includes at least the ultrasonic transducer 10 and an apparatus body 1 that generates an ultrasonic image while controlling driving of the ultrasonic transducer 10.

The ultrasonic transducer 10 may be provided in an ultrasonic probe that contacts the inspection object or contacts the inspection object through a contact medium and transmits and receives the ultrasonic wave to and from the inspection object, or in a catheter that is inserted into the inspection object and transmits and receives the ultrasonic waves to and from the inspection object. The ultrasonic transducer 10 provided in the ultrasonic imaging apparatus 100 is configured such that the vibration space of the cavity layer constituting the membrane is directly sandwiched between the upper and lower electrodes, and the thickness of the vibration space is smaller than that of the insulating layer disposed around the vibration space. Therefore, for example, at least one of Embodiments 1 to 3 described above is used for the ultrasonic transducer 10.

The apparatus body 1 includes a transmitter 12 that transmits an electrical signal for transmission to the ultrasonic transducer 10, a receiver 13 that receives an ultrasonic signal of a reflected wave from the inspection object received by the ultrasonic transducer 10, and a controller 11 that controls an operation of each part, a signal processor 15 that is provided in the controller 11 and processes the signal received by the receiver 13 to create the image or perform various calculations, and a storage 16.

The apparatus body 1 further includes an input unit 17 for an operator of the ultrasonic imaging apparatus to input operation conditions of the ultrasonic imaging apparatus to the controller 11, and a display 14 that displays processing results and the like of the signal processor 15. The input unit 17 and the display 14 may function as a user interface that allows the operator to interactively operate the apparatus body 1.

Since configuration of components constituting the apparatus body 1 is the same as that of a well-known ultrasonic imaging apparatus, description thereof will be omitted here.

Here, the operation of the ultrasonic imaging apparatus 100 will be described. First, the electrical signal for transmission is transmitted from a beamformer of the transmitter 12 to the electrodes 103 and 107 of the ultrasonic transducer 10 through a digital analog (D/A) converter (not shown), and the ultrasonic wave is transmitted from the ultrasonic transducer 10 toward the inspection object. The acoustic signal reflected in the process of propagating inside the inspection object is received by the ultrasonic transducer 10, converted into the electrical signal, passes through an A/D converter (not shown), and sent to a receiving beamformer of the receiver 13 as receiving data. The receiving beamformer performs addition processing in consideration of a time delay that the transmission takes during transmission on signals received by a plurality of elements. The received signal after the addition processing is then subjected to processing such as attenuation correction by a corrector (not shown), and then sent to the signal processor 15 as RF data. The signal processor 15 creates the image using the RF data.

The ultrasonic imaging apparatus 100 of the present invention includes the ultrasonic transducer configured such that the vibration space of the cavity layer constituting the membrane is directly sandwiched between the upper and lower electrodes, and the thickness of the vibration space is smaller than the thickness of the insulating layer disposed around the vibration space, and thus it is possible to provide the ultrasonic imaging apparatus capable of highly sensitive ultrasonic imaging. Therefore, the ultrasonic imaging apparatus 100 can also be used as an imaging apparatus that performs intravascular ultrasound (IVUS) imaging, intravascular photoacoustic (IVPA) imaging, or the like that requires highly sensitive imaging.

The ultrasonic transducer 10 may be applied to the ultrasonic imaging apparatus in which an ultrasonic transmitting probe and an ultrasonic receiving probe are provided as separate components. Specifically, since the ultrasonic receiving probe does not need to vibrate the membrane greatly to transmit the ultrasonic wave, the ultrasonic transducer 10 of Embodiments 1 to 3 having a short distance between the upper and lower electrodes and a high sensitivity is usefully applied to the ultrasonic receiving probe.

What is claimed is:

1. An ultrasonic transducer comprising:
a pair of upper and lower electrodes;
a cavity layer having a vibration space directly sandwiched between the pair of electrodes; and
an insulating layer sandwiched between the pair of electrodes and disposed around the vibration space, wherein
a vertical thickness of the insulating layer is greater than that of the cavity layer.

2. The ultrasonic transducer according to claim 1, wherein at least one of the pair of electrodes has a stepped shape so that a distance between the pair of electrodes arranged at a position vertically sandwiching the cavity layer is smaller than a distance between the pair of electrodes arranged at a position vertically sandwiching the insulating layer.

3. The ultrasonic transducer according to claim 2, wherein
the lower electrode of the pair of electrodes has a flat shape, and
the upper electrode of the pair of electrodes has a stepped shape so that a region disposed on an upper surface of the vibration space is lower than a region disposed on an upper surface of the insulating layer.

4. The ultrasonic transducer according to claim 2, wherein the cavity layer has a non-vibration space along the stepped shape between the electrode having a stepped shape and the insulating layer.

5. The ultrasonic transducer according to claim 2, wherein
the upper electrode of the pair of electrodes has a flat shape, and
the lower electrode of the pair of electrodes has a stepped shape so that a region disposed on a lower surface of the vibration space is higher than a region disposed on a lower surface of the insulating layer.

6. The ultrasonic transducer according to claim 1, wherein the thickness of the cavity layer is several nm or more and several hundred nm or less, and the thickness of the insulating layer is 5 times or more the thickness of the cavity layer.

7. The ultrasonic transducer according to claim 1, wherein the ultrasonic transducer has a plurality of transducer elements arranged therein and the transducer element comprises:
a pair of upper and lower electrodes;
a cavity layer having a vibration space directly sandwiched between the pair of electrodes; and
an insulating layer sandwiched between the pair of electrodes and disposed around the vibration space, wherein
a vertical thickness of the insulating layer is greater than that of the cavity layer.

8. A method for manufacturing an ultrasonic transducer, comprising:
a step of laminating a first insulating layer and a first electrode in this order on a substrate;
a step of laminating a sacrificial layer and a second insulating layer on the first electrode;
a step of laminating a second electrode that covers the sacrificial layer and the second insulating layer;
a step of laminating a third insulating layer on the second electrode;
a step of forming a through-hole penetrating from the third insulating layer to the sacrificial layer;
a step of forming a cavity layer by etching away the sacrificial layer through the through-hole; and
a step of embedding an insulating material in the through-hole, wherein
the step of laminating the sacrificial layer and the second insulating layer is the step of laminating them so that a vertical thickness of the second insulating layer is greater than that of the sacrificial layer.

9. The method for manufacturing the ultrasonic transducer according to claim 8, wherein
the step of laminating the sacrificial layer and the second insulating layer comprises:
a step of forming the sacrificial layer on the first electrode;
a step of forming the second insulating layer on the sacrificial layer; and
a step of removing the second insulating layer disposed over a region forming a vibration space directly sandwiched between the first electrode and the second electrode of the cavity layer out of the formed second insulating layer.

10. The method for manufacturing the ultrasonic transducer according to claim 8, wherein
the step of laminating the sacrificial layer and the second insulating layer comprises:
a step of forming the second insulating layer on the first electrode;
a step of removing the second insulating layer disposed in a region forming a vibration space directly sandwiched between the first electrode and the second electrode of the cavity layer out of the formed second insulating layer; and
a step of forming the sacrificial layer so as to cover the first electrode exposed by removing the second insulating layer and the second insulating layer.

11. The method for manufacturing the ultrasonic transducer according to claim 8, wherein the first insulating layer is formed to have a stepped shape in which a region thereof below a region directly sandwiching the sacrificial layer between the first electrode and the second electrode is higher in height from the substrate than a region thereof other than below the region directly sandwiching the sacrificial layer.

12. The method for manufacturing the ultrasonic transducer according to claim 11, wherein
the step of laminating the sacrificial layer and the second insulating layer comprises:
a step of applying the insulating material of the second insulating layer on the first electrode having a stepped shape along the stepped shape of the first insulating layer so that its upper surface is flat;
a step of etching the applied insulating material to form the second insulating layer so that an upper surface of an upper stage of the stepped shape of the first electrode is exposed; and
a step of forming the sacrificial layer on upper surfaces of the second insulating layer and the first electrode.

13. An ultrasonic imaging apparatus comprising the ultrasonic transducer according to claim 1.

14. The ultrasonic imaging apparatus according to claim 13, comprising:
a transmitting ultrasonic transducer that transmits an ultrasonic wave to an inspection object; and
a receiving ultrasonic transducer that receives the ultrasonic wave reflected from the inspection object, wherein
the ultrasonic transducer according to claim 1 is the receiving ultrasonic transducer.
